(19) United States
(12) Patent Application Publication
(10) Pub. No.: US 2014/0378736 A1
(43) Pub. Date: Dec. 25, 2014

(54) METHODS, SYSTEMS AND COMPUTER READABLE STORAGE MEDIA STORING INSTRUCTIONS FOR GENERATING A RADIATION THERAPY TREATMENT PLAN

(71) Applicants: Timothy H. Fox, Atlanta, GA (US); Eduard Schreibmann, Atlanta, GA (US)

(72) Inventors: Timothy H. Fox, Atlanta, GA (US); Eduard Schreibmann, Atlanta, GA (US)

(73) Assignee: EMORY UNIVERSITY, Atlanta, GA (US)

(21) Appl. No.: 14/310,020

(22) Filed: Jun. 20, 2014

Related U.S. Application Data
(60) Provisional application No. 61/837,708, filed on Jun. 21, 2013.

(51) Int. Cl. A61N 5/10

(52) U.S. Cl. CPC A61N 5/103
USPC 600/1

(57) ABSTRACT

Methods, systems and computer-readable storage media relate to generating a treatment plan for a patient having a patient record. The methods, systems, and the computer-readable storage medias according to embodiments can automatically generate a treatment plan for the patient using (reference) records from previous plans. The system may include: a comparison module configured to compare anatomical information of a plurality of reference records and the patient record; a comparison quantification module configured to determine a metric corresponding to a degree of matching between each reference record and patient record; a reference record selection module configured to select a reference record from the plurality of reference records based on the metric; and a treatment plan generation module configured to generate a treatment plan for the patient based on the selected reference record.

### CROSS REFERENCE TO RELATED APPLICATION

This application claims priority to Provisional Application Ser. No. 61/837,708 filed Jun. 21, 2013, which is hereby incorporated by reference in its entirety.

### BACKGROUND

Radiation therapy or radiotherapy generally is the medical use of high-energy radiation to control or kill malignant cells. One radiation therapy technique, volumetric modulated arc therapy (VMAT), optimizes the intensity modulated by a multi-leaf collimator distribution as delivered during rotational therapy according to the dose—volume histogram (DVH) constraints established by the planner in a trade-off between sparing normal tissue and irradiation the tumor. However, the attainable DVH objectives for a patient-specific anatomy are generally not known before planning. The DVHs can depend primarily on the anatomical location of the tumor relative to critical organs, with the planner modifying the energy, arc orientations and MLC configurations together with the DVH constraints until the plan is considered clinically acceptable. Thus, it is generally unknown if the plan is optimal on the given patient anatomy until a complex multi-objective optimization is performed, for example, to deduce the Pareto front, defining the family of truly optimal plans where the dose to one objective cannot be decreased without degrading another one. Additionally, treatment planning for radiation therapy, such as VMAT, can be a lengthy process that requires many rounds of optimizations to obtain the best treatment settings and optimization constraints for a given patient geometry.

### SUMMARY

Thus, there is a need for an automatic treatment planning system and methods for generating a treatment plan for radiation therapy.

The disclosure relates to systems, methods, and computer-readable media storing instructions for automatically generating a treatment plan for a patient. In some embodiments, the system may include a comparison module configured to compare anatomical information of a plurality of reference records and the patient record; a comparison quantification module configured to determine a metric corresponding to a degree of matching between each reference record and patient record; a reference record selection module configured to select a reference record from the plurality of reference records based on the metric; and a treatment plan generation module configured to generate a treatment plan for the patient based on the selected reference record.

In some embodiments, the system may further include a reference treatment database, the reference treatment database may store a plurality of reference records. Each reference record includes treatment information, anatomical information, and non-anatomical information. In some embodiments, the system may include a reference record query module configured to search the reference treatment database and obtain reference records based on search criteria.

In some embodiments, the comparison module may be configured to align the anatomical information of each reference record to the anatomical information of the patient record. In some embodiments, the anatomical information for each reference record may include segmented images of a target. In some embodiments, the patient record may include anatomical information, the anatomical information including segmented images of a target. In some embodiments, the comparison module may be configured to align a center of mass of the target of the patient record and the target of each reference record.

In some embodiments, the comparison quantifier module may be configured to determine the metric based on a distance between surfaces of the target of each reference record and of the target of the patient record. In some embodiments, the comparison quantifier module may be configured to compare regions of the target for each reference record that received a certain amount of a prescribed dose.

In some embodiments, the metric may be a single scalar value. In some embodiments, the reference record selection module may be configured to select the reference record that has a metric of a lowest value.

In some embodiments, the treatment plan generated by the treatment plan generation module may include the treatment information from the selected reference record. In some embodiments, the system may further include a treatment plan formatter configured to format the treatment information from the selected reference record. In some embodiments, the system may further include a treatment plan processor configured to process the treatment information from the selected reference record so that a position of the target in the selected reference record corresponds to a position of the target of the patient record.

In some embodiments, the target is the prostate or brain.

In some embodiments, the disclosure relates to a system comprising a processor and a memory. The system may be configured to cause: comparing anatomical information of a plurality of reference records and the patient record; determining a metric corresponding to a degree of matching between each reference record and patient record; selecting a reference record from the plurality of reference records based on the metric; and generating a treatment plan for the patient based on the selected reference record.

In some embodiments, the disclosure relates to a method for generating a treatment plan for a patient having a patient record. The method may be performed by a computer having a processor and a memory.

In some embodiments, the method may include comparing anatomical information of a plurality of reference records and the patient record; determining a metric corresponding to a degree of matching between each reference record and patient record; selecting a reference record from the plurality of reference records based on the metric; and generating a treatment plan for the patient based on the selected reference record.

In some embodiments, the method may further include receiving the plurality of reference records from a reference treatment database based on search criteria. Each reference record may include treatment information, anatomical information, and non-anatomical information.

In some embodiments, the comparing may include aligning the anatomical information of each reference record to the anatomical information of the patient record.

In some embodiments, the anatomical information for each reference record may include segmented images of a target. The patient record may include anatomical information, the anatomical information including segmented images of a target. In some embodiments, the comparing may align a center of mass of the target of the patient record and the target of each reference record.

In some embodiments, the determining the metric may be based on a distance between surfaces of the target of each reference record and of the target of the patient record. In some embodiments, the determining the metric may compare regions of the target for each reference record that received a certain amount of a prescribed dose.

In some embodiments, the metric may be a single scalar value. The selecting may select the reference record that has a metric of a lowest value.

In some embodiments, the generated treatment plan may include the treatment information from the selected reference record. In some embodiments, the target may be a brain and/or a prostate.

In some embodiments, the disclosure may relate to a computer-readable medium. The computer readable medium may store computer-executable instructions for generating a treatment plan for a patient having a patient record. The instructions may include comparing anatomical information of a plurality of reference records and the patient record; determining a metric corresponding to a degree of matching between each reference record and patient record; and selecting a reference record from the plurality of reference records based on the metric; and generating a treatment plan for the patient based on the selected reference record. The computer-readable medium may be non-transitory.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 shows a block diagram illustrating a treatment planning system according to embodiments;

FIG. 2 shows an example of information pre-processed for a reference record from the reference database;

FIG. 3 shows a method of generating a treatment plan according to embodiments;

FIG. 4 shows a method of comparing a reference record and a patient record according to embodiments;

FIGS. 5(a) and 5(b) show examples of reference and patient records before alignment and after alignment according to embodiments, respectively;

FIG. 6 shows examples of quantified comparisons according to embodiments; and

FIG. 7 shows an example of a computing system configured to generate a treatment plan for a patient record.

### DESCRIPTION OF THE EMBODIMENTS

The following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

Current treatment response assessment methods and systems do not accurately and efficiently determine personalized absorbed dose estimates of targeted radiotherapy. This can be essential to establish fundamental dose-response relationships for efficacy and toxicity and thus treatment planning. Additionally, current approaches used to assess treatment response generally do not completely capture a tumor's response.

The methods, systems, and the computer-readable storage media according to embodiments address these deficiencies. The methods, systems, and the computer-readable storage media according to embodiments relate to automatically generating prospective radiotherapy treatment plans based on anatomical feature matching to reference treatment plans, for example, previously treated cases stored in a database. By starting from a near-optimal solution and using previous optimization constraints, time spent by a user in generating a treatment plan can be reduced. This thereby can reproduce a dosimetrist's experience.

The examples discussed below are discussed with respect to the prostate as the target or region of interest. It will be understood that the disclosure is not limited to the prostate and may be applied to other organs (i.e., targets or regions of interest). For example, the targets or other organs may include but is not limited to brain among other organs.

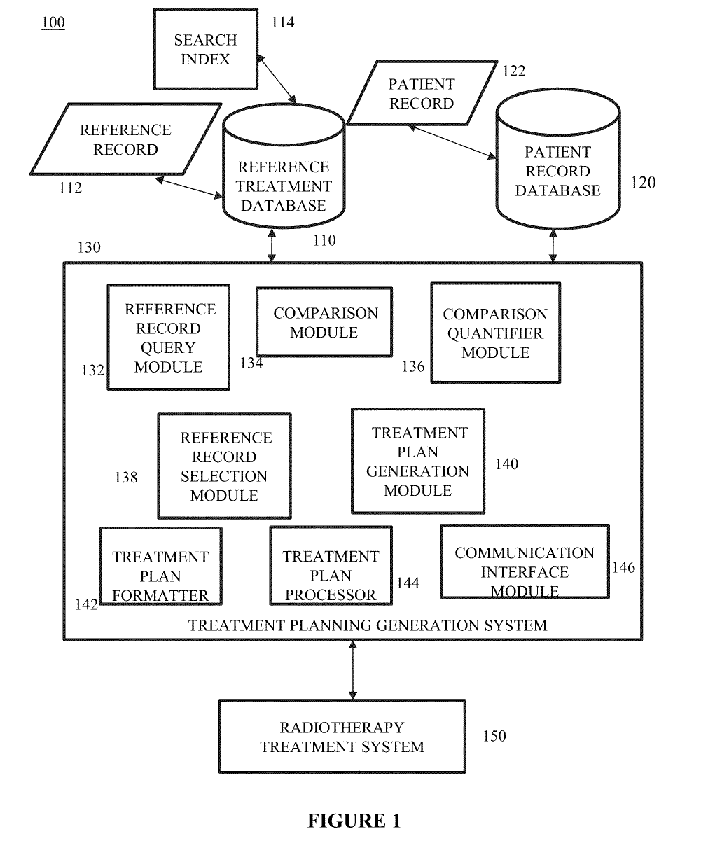

FIG. 1 shows an example of a treatment planning system capable of generating a treatment plan based on anatomical information for a patient according to embodiments. As shown in FIG. 1, the system 100 may include a reference treatment database 110. In some embodiments, the reference treatment database may include a plurality of reference records 112. The reference record may include information for a treatment plan previously used for a patient. Each reference record may include information related to a treatment plan, including but not limited to anatomical information, treatment information and non-anatomical information.

In some embodiments, the anatomical information may include but is not limited to images of a region of interest and segmented images. The treatment information may include the technique used to treat, the area treated, boost plans used, number of arcs in the plans, prescription dose, among others, as well as any combination thereof. In some embodiments, the anatomical information and the treatment information may be formatted to include images of a region of interest and associated dose matrices for an anatomy. The images may be images from any medical imaging modality, such as but not limited to MRI, CT, among others. The images may include the region of interest, the extracted geometry, as well as others.

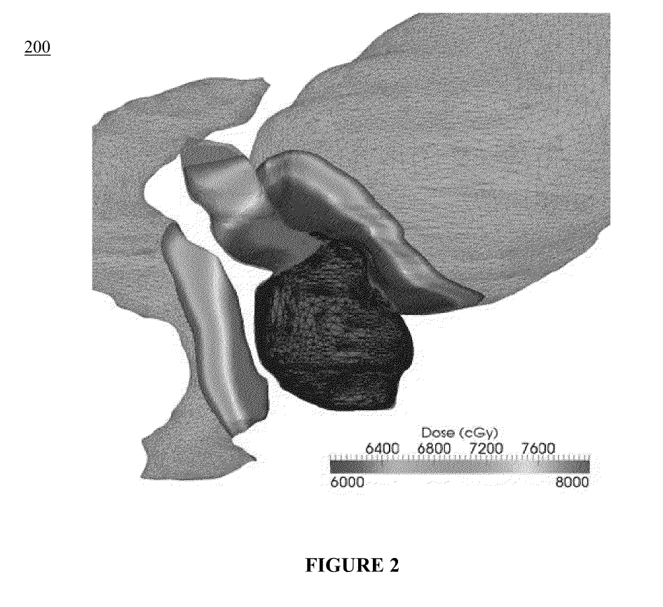

An example of anatomical information for a reference record can be found in FIG. 2. As shown in FIG. 2, the whole anatomical information for a database case is shown as a gray wireframe while the extracted geometry is shown as a solid surface color-coded with the dose delivered by the plan.

The nonanatomical information may include case information and other treatment information. The case information may include but is not limited to diagnosis, physician, institution, and other information.

In some embodiments, the information for each reference record may be linked. For example, the patient's name (non-anatomical information) may be linked to the treatment information and the anatomical information.

In some embodiments, information such as IMRT QA plans and datasets, treatment plans of the same patients for different sites, or diagnostic MRI scans associated with a patient's file may be omitted from the reference record. In some embodiments, the database 110 may include information saved in a custom format. In some embodiments, the custom format may be a search-friendly format. In some embodiments, the format may be configured to optimize speed by cropping the images and associated dose matrices to anatomy for fast access.

In some embodiments, a search index 114 may be associated with the database 110. The index 114 may store any combination of treatment, anatomical and/or non-anatomical information for each record. For example, the search index 114 may store for each record, information such as, the physician, institution, as well as treatment specific information, such as the use of boost plans, number of arcs, prescription dose, etc. As treatment options and preferences vary between institutions and physicians, non-anatomical information can be used as option to filter the result by additional physician-defined criteria. For example, records of similar anatomy to be treated with technique X may be obtained by searching for records that satisfy the condition “find cases treated with technique X.”

In some embodiments, the search index 114 may be based on the information used to create the database 110. For example, the database 110 may be created by using a DICOM query tool configured to query an institution's treatment planning system patient database. The DICOM query tool may take as input a list of patients ID and extract their corresponding images, segmentation, doses and plans as plain DICOM files. The process may include a cleaning-up step that eliminates un-needed information, such as IMRT QA plans and datasets, treatment plans of the same patients for different sites, or diagnostic MRI scans associated with a patient's file. In some embodiments, the search index 114 may be created during the cleaning process. Also, upon cleaning, the remaining data may be converted to a search-friendly format that optimizes speed by cropping the images (e.g., CT scans) and associated dose matrices to anatomy for fast access and manipulation, as well as automatically standardizing structure names.

In some embodiments, the system 100 may include a patient record database 120 that stores at least one patient record 122 for which a treatment plan may be generated. In some embodiments, the patient record database 120 may be any database, for example, a medical imaging archive, connected to a medical institution network. In some embodiments, the patient record 122 may include anatomical information, such as medical images, as well as non-anatomical information, including diagnosis, physician, and institution. The medical images may be segmented. If the medical images are not segmented, the system 100 may include a processor configured to segment the medical images according to known methods.

In some embodiments, the system 100 may include a treatment plan generation system 130 configured to generate a treatment plan for a patient record 122. The system 130 may be operated by a radiological practitioner, for example, to generate a treatment plan for the patient based on anatomical information from the reference record. The treatment plan for the patient may substantially correspond to the treatment information from the selected reference record. In this way, reference cases may be used to provide attainable DVHs and constraints to maximize the efficiency of the treatment planning process.

In some embodiments, the system 130 may include a reference record query module 132 configured to obtain reference records, for example, that have similar anatomy from a database. The reference record query module 132 may be configured to communicate with at least the reference treatment database 110. The reference record query module 132 may be configured to obtain at least one reference record 112 from the reference treatment database 110 based on search criteria. The reference record query module 132 may be configured to allow a user, for example, a physician, to specify search criteria. In some embodiments, the search criteria may substantially correspond to the search index 114. In some embodiments, the reference record query module 132 may communicate with an interface module of the reference treatment database 110.

In some embodiments, the system 130 may include a comparison module 134. The comparison module may be configured to compare each reference record obtained or received based on search criteria and the patient record. Each reference record and the patient record may be compared using a geometrical measure (a metric), for example, by comparing the anatomy (e.g., position and shape) of the target and critical organs receiving the relevant dose. In some embodiments, the comparison module may be also be configured to optimize the comparison by moving the isocenter so that the anatomy of the patient record is aligned to the isocenter of the reference record. The comparison module 134 may be configured to minimize the distance between the points defining the patient and record surfaces, for example, by using an iterative registration procedure.

In some embodiments, the comparison may be limited to high-dose regions of the reference record. Those regions may include but not limited to, for example, regions that receive 80% of the prescribed dose. In this way, irrelevant anatomy may be factored out.

In some embodiments, the system 130 may include a comparison quantifier module 136 configured to quantify the comparison by determining a metric. The metric may be a single scalar value that quantifies the degree of matching between two records. The metric compares the position and shape of target and critical organs receiving the relevant dose. In some embodiments, the metric may be a distance metric that corresponds to the similarity between the patient record and the reference record. In this way, the reference record with the closest geometrical resemblance to the new patient may be determined

The comparison quantifier module 136 may be configured to quantify the degree of matching between each reference record received from the database and the patient record by lopping through all the points defining the geometry in the reference record, recording the closest distance to the patient record, and determining the mean distance on all these points.

In some embodiments, the system 130 may include a reference record selection module 138 configured to select the reference record out of the reference records received that has the closest geometrical resemblance (e.g., highest degree of matching or metric).

In some embodiments, the system 130 may include a treatment plan generation module 140 configured to generate a treatment plan for the patient record based on the selected reference record. The treatment generation module 140 may be configured to generate a treatment plan for the patient record 122 using the treatment information (e.g., arc settings and MLC settings) corresponding to the selected reference record.

In some embodiments, the system 140 may include a treatment plan formatter 142. The treatment plan formatter 142 may be configured to format the treatment plan for the patient record, for example, in DICOM format. In some embodiments, the treatment plan formatter 142 may be configured to format the treatment plan so that it is attached to or linked to the patient record. The treatment plan may be linked as treatment information.

In some embodiments, the system 130 may include a treatment plan processor 144. The treatment plan processor 144 may be configured to further optimize and/or process the treatment plan for the patient record. In some embodiments, the treatment plan processor 144 may be configured to automatically modify the coordinates of the isocenter in the plan with the translations found by the optimization to correspond to the position of the patient on the treatment table. For example, the processor 144 may also cause the treatment plan for the patient to be imported into or communicate with another treatment planning system, for review, modification and re-optimization by the dosimetrist, if necessary.

In some embodiments, the system 130 may include a communication interface module 146 configured to conduct receiving and transmitting of data between the reference treatment database 110, the patient record database 120, and/or a radiotherapy treatment system 170, as well as other modules on the system and/or network. The communication interface module 146 may be a wired and/or wireless interface, a switched circuit wireless interface, a network of data processing devices, such as LAN, WAN, the interne, or combination thereof. The communication interface may be configured to execute various communication protocols, such as Bluetooth, wireless, and Ethernet, in order to establish and maintain communication with at least another module on the network.

The system 100 may further include a radiotherapy treatment system 150. The radiotherapy treatment system 150 may include any system capable of performing radiotherapy, including but not limited to, external beam radiation therapy (EBRT or XRT) or teletherapy, brachytherapy or sealed source radiation therapy, and systemic radioisotope therapy or unsealed source radiotherapy. In some embodiments, the treatment planning generation system 130 may output the treatment plan for a patient to the radiotherapy treatment system 150. In some embodiments, the treatment planning generation system 130 may alternatively or additionally output the treatment plan for a patient, along with the other information of the patient record, to the treatment planning database to be added as a reference record.

In some embodiments, the modules of the treatment planning system 100 may be connected to a data network, a wireless network, or any combination thereof. In some embodiments, the treatment plan generation system may be at least in part be based on cloud computing architecture. In some embodiments, the reference record database and optionally the treatment plan generation platform may be applied to a self-hosted private cloud based architecture, a dedicated public cloud, a partner-hosted private cloud, as well as any cloud based computing architecture.

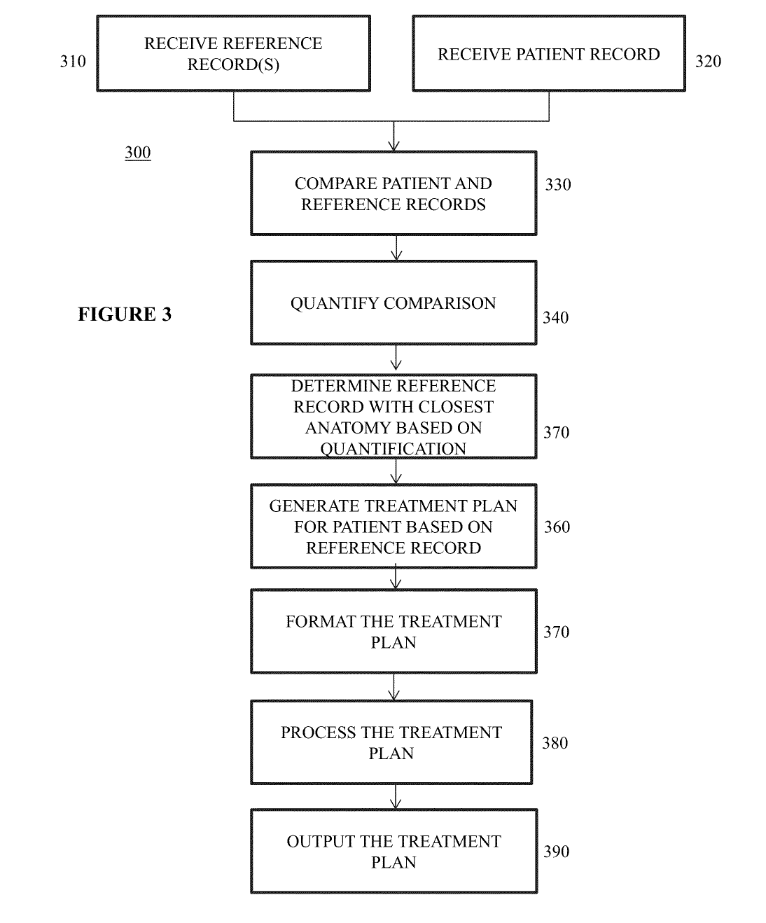

FIG. 3 illustrates a method 300 according to embodiments to generate a treatment plan for a patient record.

The methods of the disclosure are not limited to the steps described herein. The steps may be individually modified or omitted, as well as additional steps may be added.

Unless stated otherwise as apparent from the following discussion, it will be appreciated that terms such as “averaging,” “selecting,” “filtering,” “combining,” “comparing,” “segmenting,” “generating,” “aligning,” “determining,” “obtaining,” “processing,” “computing,” “selecting,” “estimating,” “formatting,” “outputting,” “calculating” “receiving,” or “acquiring,” “aligning,” “minimizing,” “quantifying,” “analyzing,” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

As shown in FIG. 3, the method 300 may include a step 310 of receiving at least one reference record based on search criteria, for example, from a reference treatment database (e.g., database 110). In some embodiments, a plurality of reference records may be received based on search criteria. In some embodiments, at least two reference records may be received based on search criteria. In some embodiment, any number of reference records may be received.

The method may include a step of receiving a patient record for which a treatment plan is to be generated (step 320). The patient record may be stored in a patient record database, e.g., database 120.

Next, the method 300 may include a step 330 of comparing the patient record and each reference record. In some embodiments, the comparing step 330 may include comparing the anatomical information (e.g., the segmented images) of the reference record to the patient record. In some embodiments, the patient record and each reference record may be compared by aligning the images of the patient and reference records.

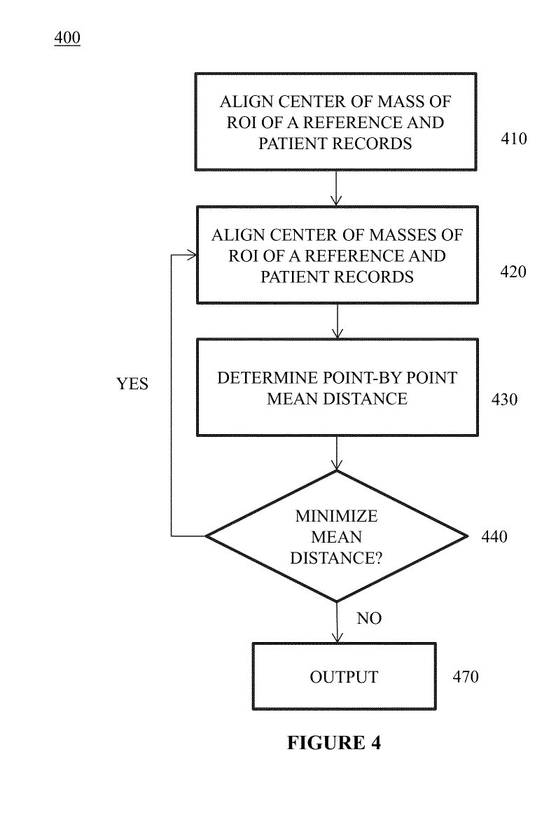

FIG. 4 shows a method 400 of comparing the anatomical information of the patient record and the reference record according to embodiments. In some embodiments, the comparison may be in terms of a score function.

As shown in FIG. 4, the method 400 may include a step 410 of determining the point that corresponds to the center (e.g., center of mass) of a region of interest in the patient record and the reference record, for example, by averaging the x coordinates, the y coordinates, and then the z coordinates.

The method 400 may then include a step 420 aligning the center of masses of the target for the reference and patient records. Next, the method 400 may include a step 430 of determining the point by point mean distance. Next, the method 400 includes a step 440 of minimizing the point by point mean distance. The step 440 can be considered an optimization step in which the translations can be the variables and the mean distance can be the metric. In each step 440, the translations may be adjusted to minimize the mean distance if certain conditions are satisfied (yes), including but not limited to the number of iterations reaches a certain prescribed limit and/or the changes in the mean distance can be considered insignificant (e.g., the changes meet a certain threshold). If it is determined that the mean-distance no longer needs to be optimized (no at step 440), the comparison may be outputted for quantification.

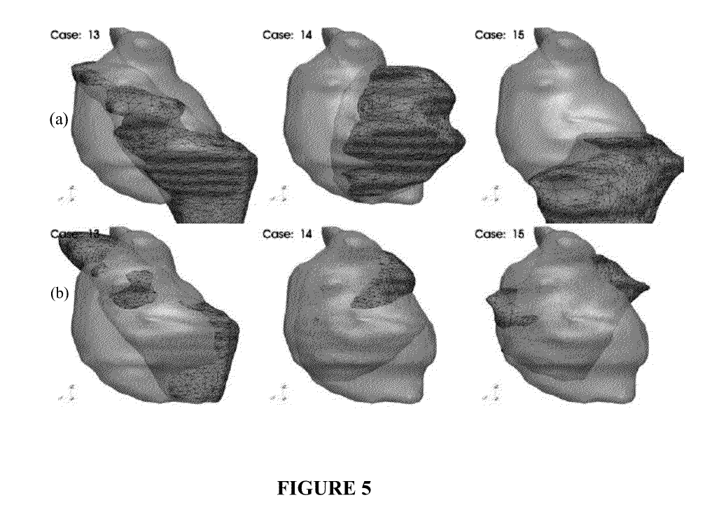

FIGS. 5(a) and 5(b) show examples of the comparison step 400. FIG. 5(a) shows a row that includes a series of three different patients and reference records (the patient's prostate is lighter gray and the reference record is darker gray) before alignment. FIG. 5(b) shows a row of the three cases after the target is aligned in the step 400 (e.g., the iterative procedure that minimizes the distance between prostrate shapes).

Next, the method 300 may include a step 340 of quantifying the comparison. The comparison (e.g., the degree of matching) may be quantified by determining the mean distance between the points defining the surfaces of the target of patient record and the reference record. The determination of the distance may be determined by lopping through all the points defining the geometry in the reference record, determining the closest distance to the patient record, and computing the mean distance on all these points. In this way, the surfaces of the target between the patient record and the reference record may be compared for similarity. The mean distance may correspond to the similarity score or metric used to select the reference record with the closest anatomy to the patient record. For example, in some embodiments, if the surfaces are identical, the mean distance corresponds to zero, and if the surfaces have different shapes, the mean distance would have a higher value.

In some embodiments, the quantification may be limited to the certain regions of the reference record, for example, those regions that may be considered high-dose regions. This region may include only points receiving a certain amount of the prescribed dose. The certain amount may include but is not limited to about 80% of the prescribed dose. In some embodiments, the certain amount may be more or less than about 80% of the prescribed dose.

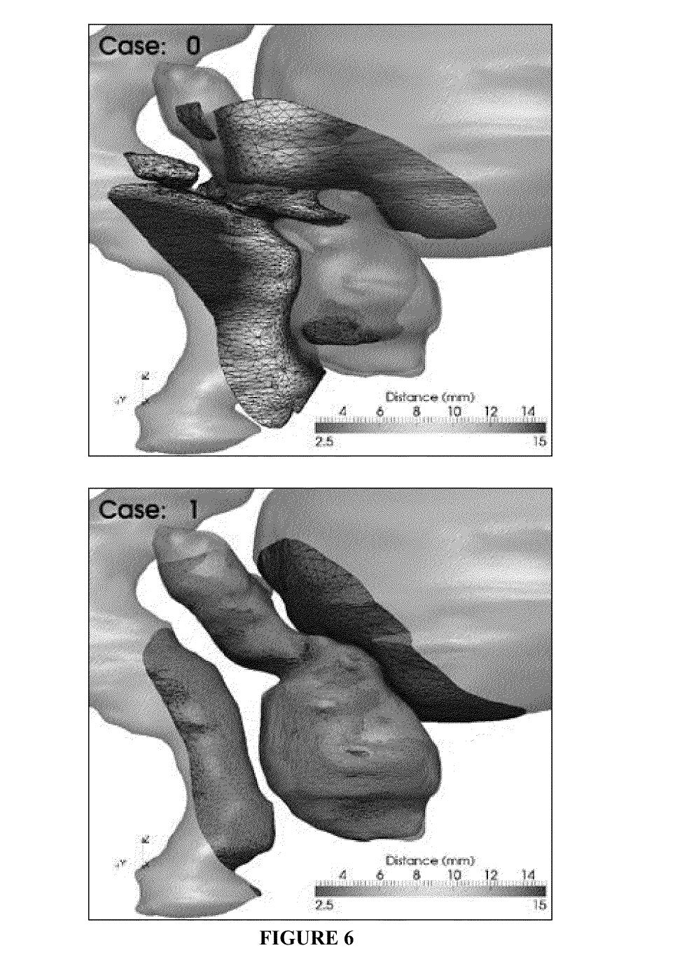

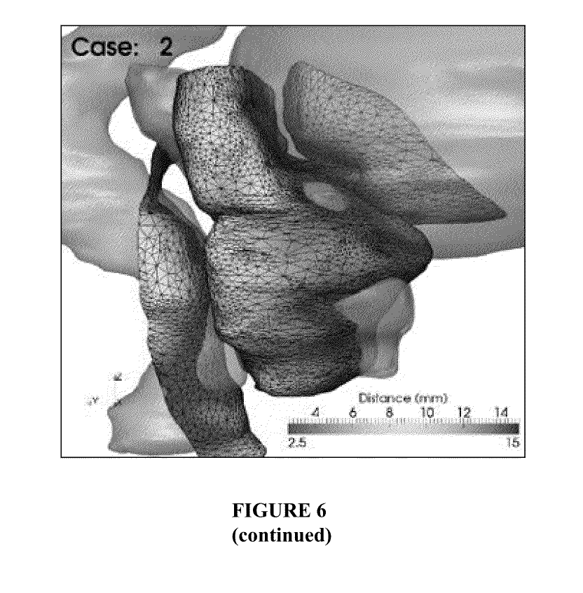

FIG. 6 shows examples 600 of quantification of the comparison between three different reference records and patient record. The comparison was scored by distance between the surfaces in the high dose region of the reference record and the patient record. The light gray surface represents the patient segmentation and the surface wireframe is the record segmentation in the high dose region color-coded with the distance to the patient record segmentation. The mean distance on this surface can be used as the metric or similarity score.

It will be understood that steps 330 through 340 may be repeated for each reference record received.

In some embodiments, the method 300 may include a step 350 of determining the reference record based on the quantification with the closest anatomy. The determining step 350 may be based on the similarity score or metric. As discussed above, if the surfaces are identical, the mean distance corresponds to zero, and if the surfaces have different shapes, the mean distance would have a higher value. In some embodiments, the determining step 350 may determine and select the reference record that has the lowest similarity score or metric.

In some embodiments, the method 300 may include a step 360 of generating a treatment plan for the patient record based on the determined (also referred to as “selected”) reference record. The treatment plan for the treatment plan may include the treatment information from the reference record. The treatment information may include but is not limited to radiation settings, constraints, machine settings, among others.

In some embodiments, the method 300 may include a step 370 of formatting the treatment plan for the patient. The formatting step 370 may include reformatting the plan into a format, such as DICOM format.

In some embodiments, the method 300 may optionally include a step 380 of processing the treatment plan for the patient record. The step 380 may occur before and/or after step 390 of outputting. The step 380 may include processing the treatment plan for the patient record. The processing may include modifying the coordinates of the isocenter in the plan with the translations found by the optimization to correspond to the position of the patient on the treatment table. For example, the processing may include causing the radiotherapy system to move the irradiation center high or lower to position to the patient's target.

In some embodiments, the step may include optimizing the treatment plan. For example, the treatment plan may be transmitted to another treatment planning system for further optimization.

In some embodiments, the method 300 may include a step 390 of outputting the treatment plan. In some embodiments, the outputting may include but is not limited to displaying the image(s), printing the image(s), and storing the image(s) remotely or locally. In other embodiments, the image(s) may be forwarded for further processing.

In some embodiments, the method may further include transmitting the generated plan to a radiotherapy treatment system and/or another treatment planning system. The method may also include transmitting the generated plan as a reference record to the reference treatment database 110.

Embodiments of the methods described herein may be implemented using computer software, hardware, firmware, special purpose processes, or a combination thereof If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure. An example of hardware for performing the described functions is detailed below.

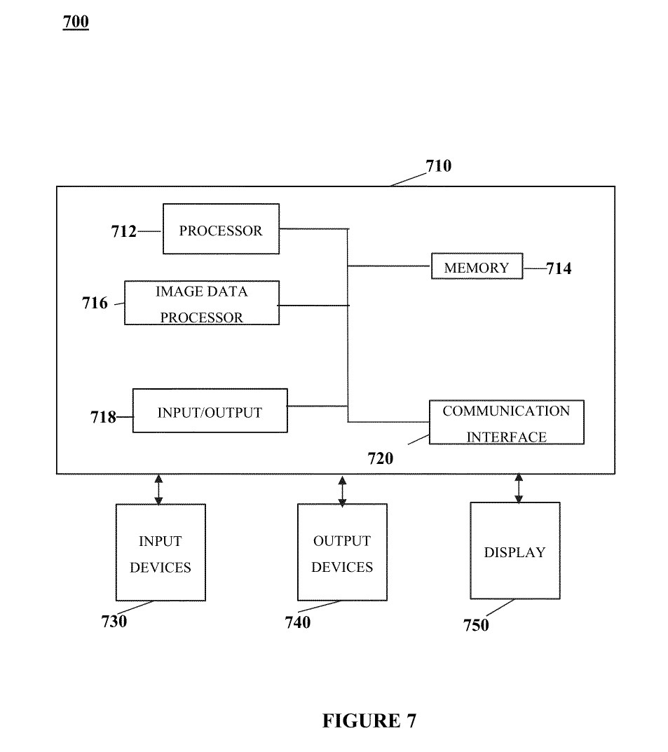

FIG. 7 illustrates an example of a computer system 700 for generating a treatment plan according to embodiments. The system for carrying out the embodiments of the methods disclosed herein is not limited to the system shown in FIG. 7. Other systems may be used.

The system 700 may include any number of modules that communicate with other through electrical or data connections (not shown). In some embodiments, the modules may be connected via a wired network, wireless network, or combination thereof. In some embodiments, the networks may be encrypted. In some embodiments, the wired network may be, but is not limited to, a local area network, such as Ethernet, or wide area network. In some embodiments, the wireless network may be, but is not limited to, any one of a wireless wide area network, a wireless local area network, a Bluetooth network, a radio frequency network, or another similarly functioning wireless network.

It is also to be understood that the system may omit any of the modules illustrated and/or may include additional modules not shown. It is also be understood that more than one module may be part of the system although one of each module is illustrated in the system. It is further to be understood that each of the plurality of modules may be different or may be the same. It is also to be understood that the modules may omit any of the components illustrated and/or may include additional component(s) not shown.

In some embodiments, the modules provided within the system may be time synchronized. In further embodiments, the system may be time synchronized with other systems, such as those systems that may be on the medical facility network.

The system 700 may be a computing system, such as a workstation, computer, or the like. The system 700 may include one or more processors 712. The processor(s) 712 (also referred to as central processing units, or CPUs) may be any known central processing unit, a processor, or a microprocessor. The CPU 712 may be coupled directly or indirectly to one or more computer-readable storage media (e.g., memory) 714. The memory 714 may include random access memory (RAM), read only memory (ROM), disk drive, tape drive, etc., or a combinations thereof The memory may also include a frame buffer for storing image data arrays.

The CPU 712 may be configured to generate a treatment plan. In some embodiments, the CPU 712 may be capable of performing the image processing functionality. In other embodiments, the system may include a separate CPU for performing the image processing functionality.

In some embodiments, the CPU 712 may be configured to process the information d from the patient record and/or reference record. In some embodiments, the system 710 may include one or more image processors 716 (e.g., any known processing unit such as a CPU, a processor, or a microprocessor) configured to process raw image data. The processed data and results may then be stored in the memory 714. In some embodiments, another computer system may assume the image analysis or other functions of the CPU 712 or image processor 716. In response to commands received from the input device, the image data stored in the memory 714 may be archived in long term storage or may be further processed by the image processor and presented on a display.

In some embodiments, the disclosed methods (e.g., FIGS. 3 and 4) may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system. In addition, various other peripheral devices may be connected to the computer platform such as an additional data storage device, a printing device, and other I/O (input/output) devices.

In some embodiments, the system 710 may include a communication interface 720 configured to conduct receiving and transmitting of data between other modules on the system and/or network. The communication interface 720 may be a wired and/or wireless interface, a switched circuit wireless interface, a network of data processing devices, such as LAN, WAN, the internet, or combination thereof The communication interface may be configured to execute various communication protocols, such as Bluetooth, wireless, and Ethernet, in order to establish and maintain communication with at least another module on the network.

In some embodiments, the system 710 may include an input/output interface 718 configured for receiving information from one or more input devices 730 (e.g., a keyboard, a mouse, and the like) and/or conveying information to one or more output devices 740 (e.g., a printer, a CD writer, a DVD writer, portable flash memory, etc.). In some embodiments, the one or more input devices 730 may configured to control the generation of the images, display of images on a display 750, and/or printing of the images by a printer interface.

It is to be understood that the embodiments of the disclosure be implemented in various forms of hardware, software, firmware, special purpose processes, or a combination thereof In one embodiment, the disclosure may be implemented in software as an application program tangible embodied on a computer readable program storage device. The application program may be uploaded to, and executed by, a machine comprising any suitable architecture. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that, because some of the constituent system components and method steps depicted in the accompanying figures can be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims

What is claimed:

1. A system configured to generate a radiological treatment plan for a patient having a patient record, comprising:
a comparison module configured to compare anatomical information of a plurality of reference records and the patient record;
a comparison quantification module configured to determine a metric corresponding to a degree of matching between each reference record and patient record;
a reference record selection module configured to select a reference record from the plurality of reference records based on the metric; and
a treatment plan generation module configured to generate a treatment plan for the patient based on the selected reference record.

2. The system according to claim 1, further comprising:
a reference treatment database, the reference treatment database storing a plurality of reference records,
wherein each reference record includes treatment information, anatomical information, and non-anatomical information.

3. The system according to claim 1, further comprising:
a reference record query module configured to search the reference treatment database and obtain reference records based on search criteria.

4. The system according to claim 2, wherein the comparison module is configured to align the anatomical information of each reference record to the anatomical information of the patient record.

5. The system according to claim 4, wherein:
the anatomical information for each reference record includes segmented images of a target;
the patient record includes anatomical information, the anatomical information including segmented images of a target; and
the comparison module is configured to align a center of mass of the target of the patient record and the target of each reference record.

6. The system according to claim 5, wherein:
the comparison quantifier module is configured to determine the metric based on a distance between surfaces of the target of each reference record and of the target of the patient record.

7. The system according to claim 1, wherein:
the comparison quantifier module is configured to compare regions of the target for each reference record that received a certain amount of a prescribed dose.

8. The system according to claim 1, wherein:
the metric is a single scalar value; and
the reference record selection module is configured to select the reference record that has a metric of a lowest value.

9. The system according to claim 1, wherein:
the treatment plan generated by the treatment plan generation module includes the treatment information from the selected reference record.

10. The system according to claim 1, wherein the target is the prostate.

11. A method for generating a treatment plan for a patient having a patient record, comprising:
comparing anatomical information of a plurality of reference records and the patient record;
determining a metric corresponding to a degree of matching between each reference record and patient record;
selecting a reference record from the plurality of reference records based on the metric; and
generating a treatment plan for the patient based on the selected reference record.

12. The method according to claim 11, further comprising:
receiving the plurality of reference records from a reference treatment database based on search criteria,
wherein each reference record includes treatment information, anatomical information, and non-anatomical information.

13. The method according to claim 12, wherein the comparing includes aligning the anatomical information of each reference record to the anatomical information of the patient record.

14. The method according to claim 13, wherein:
the anatomical information for each reference record includes segmented images of a target;
the patient record includes anatomical information, the anatomical information including segmented images of a target; and
the comparing aligns a center of mass of the target of the patient record and the target of each reference record.

15. The method according to claim 11, wherein:
the determining the metric is based on a distance between surfaces of the target of each reference record and of the target of the patient record.

16. The method according to claim 15, wherein:
the determining the metric compares regions of the target for each reference record that received a certain amount of a prescribed dose.

17. The method according to claim 11, wherein:
the metric is a single scalar value; and
the selecting selects the reference record that has a metric of a lowest value.

18. The method according to claim 11, wherein:
the generated treatment plan includes the treatment information from the selected reference record.

19. The method according to claim 11, wherein the target is the prostate.

20. A computer readable medium storing computer-executable instructions for generating a treatment plan for a patient having a patient record, the instructions including:
comparing anatomical information of a plurality of reference records and the patient record;
determining a metric corresponding to a degree of matching between each reference record and patient record;
selecting a reference record from the plurality of reference records based on the metric; and
generating a treatment plan for the patient based on the selected reference record.
